(19) United States
(12) Patent Application Publication
(10) Pub. No.: US 2017/0057157 A1
(43) Pub. Date: Mar. 2, 2017

(54) Bonding Apparatus and Method

(71) Applicant: The Procter & Gamble Company, Cincinnati, OH (US)

(72) Inventor: Stuart Lebowitz, Deerfield Township, OH (US)

(21) Appl. No.: 15/242,850

(22) Filed: Aug. 22, 2016

Related U.S. Application Data
(60) Provisional application No. 62/209,386, filed on Aug. 25, 2015.

(51) Int. Cl. B29C 65/48; A61F 13/49; A61F 13/15; B29C 65/08

(52) U.S. Cl. CPC B29C 65/48; B29C 65/08; A61F 13/49; A61F 13/15577; B29L 2031/4878

(57) ABSTRACT

The present disclosure relates to methods and apparatuses for mechanically modifying substrates. The apparatuses herein may include a tool roll and an anvil roll. The tool roll may include tooling members adapted to perform various different operations, such as bonding, cutting, embossing, and/or activation of advancing substrates. The tooling members and associated tooling surfaces may not extend completely around the circumference of the tool roll. As such, the tool roll also includes one or more bearer rings that engage the anvil roll during portions of the tool roll rotation where the tooling members are not in engagement with the anvil roll. The tooling members and bearer rings herein are configured to help reduce vibrations in the anvil and/or tool roll caused by the alternating engagements of the tooling surfaces and bearer rings with the anvil roll during rotation.

### CROSS REFERENCE TO RELATED APPLICATIONS

This application claims the benefit of U.S. Provisional Application No. 62/209,386 filed on Aug. 25, 2015, which is incorporated herein by reference.

### FIELD OF THE INVENTION

The present disclosure relates to methods for manufacturing absorbent articles, and more particularly, to apparatuses and methods for bonding substrates that may be used as components of absorbent articles.

### BACKGROUND OF THE INVENTION

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from advancing web or webs are combined with other individual components created from other advancing web or webs. Webs of material and component parts used to manufacture diapers may include: backsheets, topsheets, leg cuffs, waist bands, absorbent core components, front and/or back ears, fastening components, and various types of elastic webs and components such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

During the assembly process, various components and/or advancing webs of material may be bonded together in various ways. For example, in some processes, advancing webs and/or components may be bonded together with adhesives. In other processes, advancing webs and/or components may be mechanically bonded together with heat and/or pressure without the use of adhesives. An example of such a mechanical bonding method and apparatus is disclosed in U.S. Pat. No. 4,854,984, wherein two laminae are bonded together by advancing through a nip between a rotating anvil cylinder and a rotating patterned cylinder that includes one or more pattern elements. The patterned cylinder and anvil cylinder are biased toward each other by applying a force on the patterned cylinder and/or the anvil cylinder to create a desired bonding pressure between the pattern elements and the anvil roll. As the patterned cylinder and anvil cylinder rotate, the pattern elements on the patterned cylinder exert pressure on the two laminae against the anvil roll to create discrete bond sites. In some bonding configurations, the pattern elements do not extend entirely around the outer circumference of the patterned cylinder. Thus, the patterned cylinder may include a bearer ring that engages the anvil cylinder to help maintain relative positions between the pattern cylinder and anvil cylinder during the periods of rotation when no pattern elements engage the anvil cylinder.

Depending on the how the pattern elements and/or bearer rings are constructed, the patterned cylinder and/or the anvil cylinder may be displaced by different amounts during rotation depending on whether the pattern elements or the bearer rings are engaging the anvil. In turn, the different displacements can cause relatively high vibrations as the patterned cylinder and anvil rotate. As such, relatively high vibration can result in anvil surface and/or pattern element wear, chipping, spalling, buckling, and/or otherwise fracturing, referred to generally as bond tool breakdown, sometimes necessitating frequent and costly repairs. Excessive vibration may also damage the laminae by forming holes and/or tears in or around the bond sites. For example, pattern elements may become deformed and/or fail after prolonged use. As a pattern element degrades, the bonds created thereby may also have inconsistent aesthetic appearances; have relatively weaker strengths; and may tear or cut the bonded laminae in areas adjacent to the bonds.

Consequently, it would be beneficial to provide a method and apparatus for mechanically bonding substrates configured to operate with relatively low vibration during rotation of the pattern cylinder and anvil.

### SUMMARY OF THE INVENTION

The present disclosure relates to methods and apparatuses for mechanically modifying substrates. The apparatuses herein may include a tool roll and an anvil roll. The tool roll may be configured in various ways and may include tooling members adapted to perform various different operations, such as bonding, cutting, embossing, and/or activation of advancing substrates. In some configurations, the tooling members and associated tooling surfaces may not extend completely around the circumference of the tool roll. As such, the tool roll also includes one or more bearer rings that engage the anvil roll during portions of the tool roll rotation where the tooling members are not in engagement with the anvil roll. The tooling members and bearer rings herein are configured to help reduce vibrations in the anvil and/or tool roll caused by the alternating engagements of the tooling surfaces and bearer rings with the anvil roll during rotation.

In one form, an apparatus for bonding substrates comprises: a frame; an anvil roll rotatably connected with the frame and adapted to rotate about a first axis of rotation, the anvil roll comprising an outer circumferential surface; a bonding roll rotatably connected with the frame and adapted to rotate about a second axis of rotation, wherein the first axis of rotation is parallel with the second axis of rotation, the bonding roll comprising: a bearer ring comprising a bearer surface extending from a first end to a second end to define a circumferential length, the bearer ring extending for less than 360 degrees around the second axis of rotation to define a circumferential gap region between the first end and the second end, the bearer ring comprising a contact stiffness, Kb; a pattern element comprising a pattern surface circumferentially offset from the bearer ring and positioned in the circumferential gap region, the pattern element comprising a contact stiffness Kp; wherein the bonding roll and the anvil roll are biased toward each other with a force F, such that as the anvil roll and bonding roll rotate, a pattern pressure Pp is created between the pattern surface and the outer circumferential surface of the anvil roll and a bearer pressure Pb is created between the bearer surface and the outer circumferential surface of the anvil roll; and wherein the force F displaces the pattern surface by a distance Xp, wherein Xp is defined by F/Kp and wherein the force F displaces the bearing surface by a distance Xb, wherein Xb is defined by F/Kb; and wherein Kp is about equal to Kb.

In another form, an apparatus for bonding substrates comprises: a frame; an anvil roll rotatably connected with the frame and adapted to rotate about a first axis of rotation, the anvil roll comprising an outer circumferential surface; a bonding roll rotatably connected with the frame and adapted to rotate about a second axis of rotation, wherein the first axis of rotation is parallel with the second axis of rotation, the bonding roll comprising: a first bearer ring comprising a first bearer surface extending from a first end to a second end to define a circumferential length, the first bearer ring extending for less than 360 degrees around the second axis of rotation; a second bearer ring comprising a second bearer surface extending from a first end to a second end to define a circumferential length, the second bearer ring extending for less than 360 degrees around the second axis of rotation; the second end of the first bearer surface circumferentially offset from the first end of the second bearer surface to define a circumferential gap region between the first end and the second end; a pattern element comprising a pattern surface circumferentially offset from the first and second bearer rings and positioned in the circumferential gap region; wherein the bonding roll and the anvil roll are biased toward each other with a force F, such that as the anvil roll and bonding roll rotate, a pattern pressure Pp is created between the pattern surface and the outer circumferential surface of the anvil roll and bearer pressures Pb are created between the first and second bearer surfaces and the outer circumferential surface of the anvil roll; and wherein the force F displaces the pattern surface by a distance Xp; wherein the force F displaces the first bearer surface and the second bearer surface by a distance Xb; and wherein Xb is about equal to Xp.

In yet another form, a method of bonding substrates comprises the steps of: providing an anvil roll adapted to rotate about a first axis of rotation, the anvil roll comprising an outer circumferential surface; providing a bonding roll adapted to rotate about a second axis of rotation adjacent the anvil roll, wherein the first axis of rotation is parallel with the second axis of rotation, the bonding roll comprising a bearer surface and a pattern surface; rotating the anvil roll and the bonding roll in opposite directions such that the pattern surface and the bearer surface alternatingly contact the outer circumferential surface of the anvil roll; biasing the anvil roll and the bonding roll toward each other with a force, F; displacing the pattern surface by a distance Xp with the force F; displacing the bearer surface by a distance Xb with the force F, wherein Xb is about equal to Xp; and advancing a first substrate and a second substrate in a machine direction between the bonding roll and the anvil roll to form a discrete bond region between the first and second substrates.

### BRIEF DESCRIPTION OF THE DRAWINGS

FIG. 1 is a schematic side view of a bonding apparatus.

FIG. 2 is a front side view of a bonding apparatus.

FIG. 3 is a detailed view of a pattern roll.

FIG. 4 is a cross-sectional view of the pattern roll of FIG. 3 taken along line 4-4.

FIG. 4A is a side of a pattern roll with more than one bearer rings circumferentially extending around an axis of rotation.

FIG. 5 is a cross-sectional view of the pattern roll and bearer ring of FIG. 4 taken along line 5-5.

FIG. 6 is a detailed view of a pattern element.

FIG. 7 is a view of a bonded laminate from FIG. 1 taken along line 7-7.

FIG. 8 is a cross-sectional view of the bonded laminate and a discrete bond region from FIG. 7 taken along line 8-8.

FIG. 9A is a schematic representation of a bonding apparatus in terms of force, displacements, and contact stiffness of pattern elements.

FIG. 9B is a schematic representation of a bonding apparatus in terms of force, displacements, and contact stiffness of the bearer rings.

FIG. 10 is a graphic representation of the amplitude of the vibrations displacements change as a function of the different contact stiffness Kb and Kp during rotation of the pattern roll.

FIG. 11A is a partially cut away plan view of a diaper with the portion of the diaper that faces away from a wearer oriented towards the viewer.

FIG. 11B is a plan view of the diaper of FIG. 11A with the portion of the diaper that faces toward a wearer oriented towards the viewer.

### DETAILED DESCRIPTION OF THE INVENTION

The following term explanations may be useful in understanding the present disclosure:

“Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The present disclosure relates to methods and apparatuses for manufacturing absorbent articles, and in particular, to methods and apparatuses for mechanically modifying substrates. The apparatuses herein may include a tool roll and an anvil roll, wherein the tool roll may include one or more tooling members protruding radially outward, wherein each tooling member includes a tooling surface adapted to engage and modify a substrate advancing between the anvil roll and the tool roll. As discussed below, the tool roll may be configured in various ways to perform various different operations, such as bonding, cutting, embossing, and/or activation of advancing substrates. In some embodiments, the tooling members and associated tooling surfaces may not extend completely around the circumference of the tool roll. As such, the tool roll also includes one or more bearer rings that engage the anvil roll during portions of the tool roll rotation where the tooling members are not in engagement with the anvil roll. The tooling members and bearer rings herein are configured to help reduce vibrations in the anvil and/or tool roll caused by the alternating engagements of the tooling surfaces and bearer rings with the anvil roll during rotation. More particularly, the tool roll and the anvil roll are biased toward each other with a force F, and during rotation of the tool roll and anvil roll, the force F displaces the tooling surface by a first distance and displaces the bearer surface by a second distance. As discussed in more detail below, configuring the contact stiffness of the tooling members and bearer rings to reduce the difference between the magnitude of the first and second distances helps to reduce vibrations. In turn, relatively low vibrations may help reduce the frequency of tooling member damage, including buckling, deformations, dulling, and/or failures and may help provide for more relatively more consistent tooling operations.

It is to be appreciated that various arrangements and configurations of the apparatuses and methods herein may be used to machine and/or modify various types of advancing substrates. For example, as discussed in more detail below, apparatuses and methods according to the present disclosure may be utilized to bond various substrates together during the production of various components of absorbent articles, such as diapers.

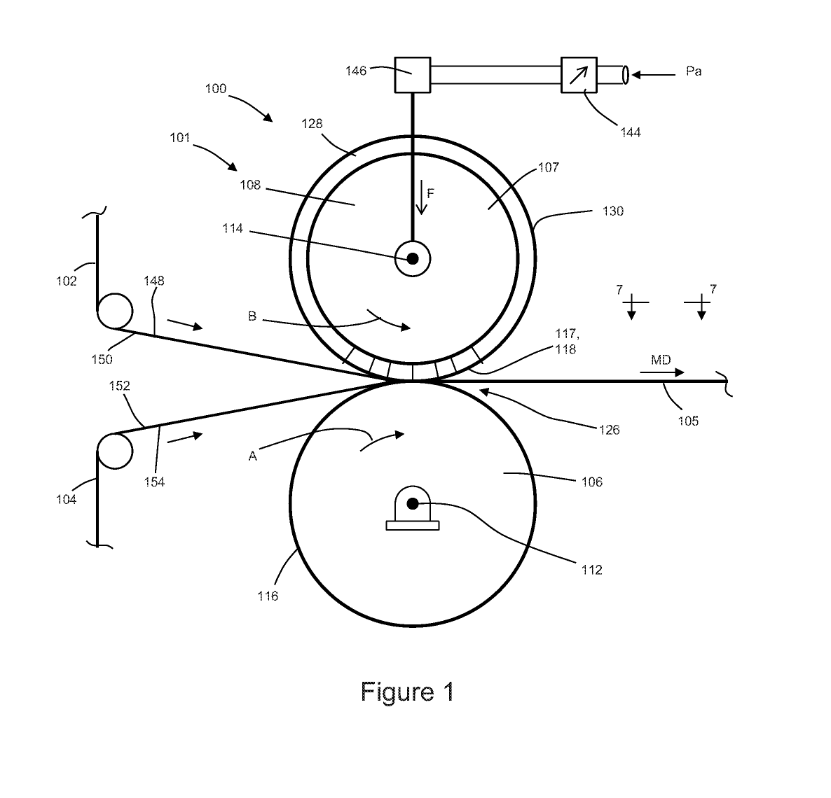

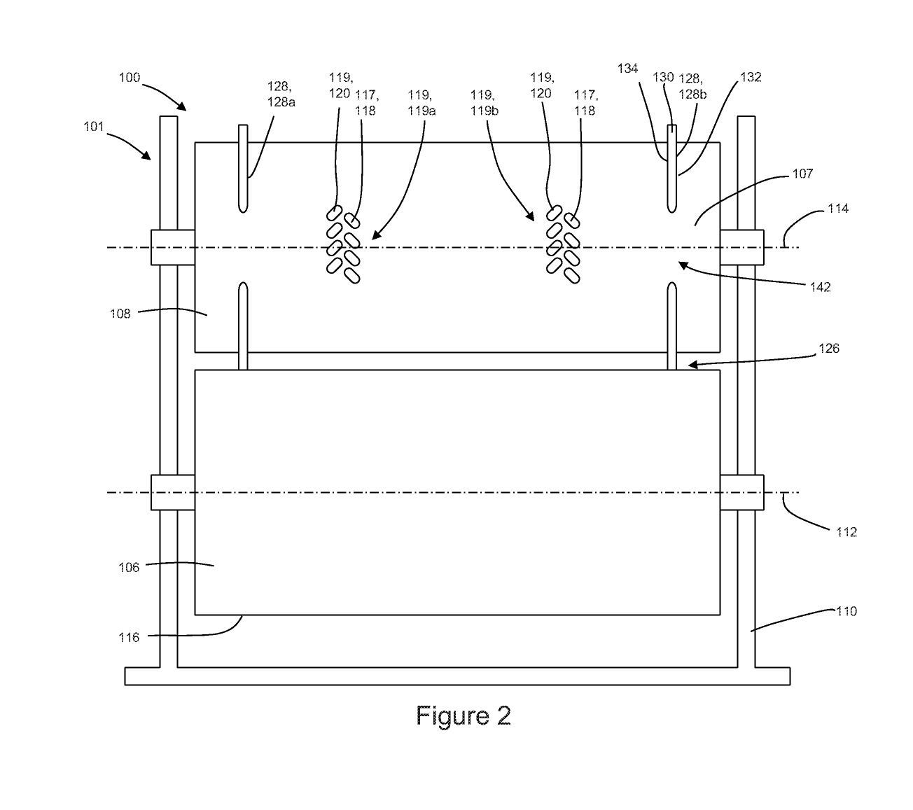

FIGS. 1 and 2 show an embodiment of a tooling apparatus 100 configured as a bonding apparatus 101 to bond a first substrate 102 and a second substrate 104 together to form a laminate 105. As shown in FIGS. 1 and 2, the apparatus 100 may include an anvil roll 106 and a tool roll 107 configured as a pattern roll 108, both rotatably connected with a frame 110. The anvil roll 106 is adapted to rotate in direction A around a first axis of rotation 112 and the pattern roll 108 is adapted to rotate in an opposite direction B around a second axis of rotation 114. The anvil roll 106 includes an outer circumferential surface 116. The pattern roll 108 may also include one or more tooling members 117 in the form of one or more pattern elements 118, each pattern element 118 including a tooling surface 119 in the form of a pattern surface 120. With particular reference to FIG. 6, the pattern roll 108 may include a base circumferential surface 122, wherein each pattern element 116 includes a circumferential wall 124 that protrudes radially outward from the base circumferential surface 122 to define a distance, Hp, between the pattern surface 120 and the base surface 122. It is to be appreciated that in some embodiments, the circumferential wall 124 may be perpendicular to base circumferential surface 122 or may sloped or tapered with respect to the base circumferential surface 122. As shown in FIG. 1, the pattern roll 108 is adjacent the anvil roll 106 so as to define a nip 126 between the pattern roll 108 and the anvil roll 106, and more particularly, to define a nip 126 between the pattern surface 120 of each pattern element 118 and outer circumferential surface 116 of the anvil roll 106.

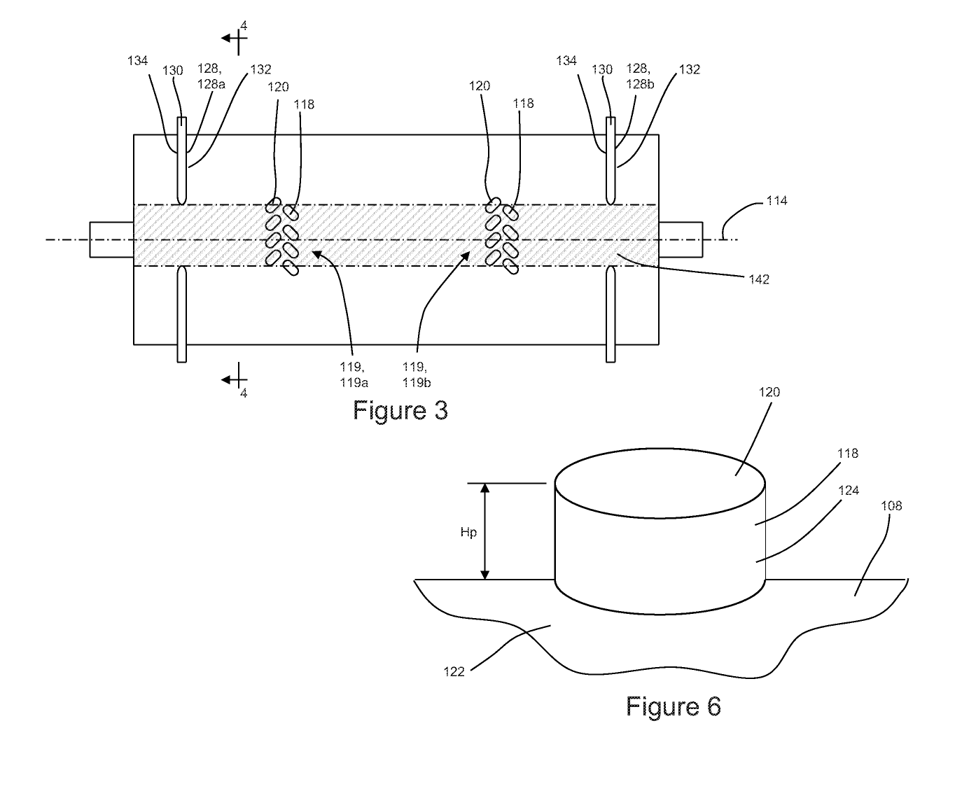

As shown in FIGS. 1 and 2, the pattern elements 118 may be arranged on the pattern roll 108 for less than 360 degrees around the axis of rotation 114. Thus, as shown in FIGS. 1 and 2, the pattern roll 108 may also include one or more bearer rings 128, each including a bearer surface 130 that engage the anvil roll 106 during portions of patter roll 108 rotation when the pattern elements 118 are not engaging the anvil roll 106. As shown in FIGS. 3-5, the bearer ring 128 may include a first sidewall 132 and a second sidewall 134 that protrude radially outward from a base circumferential surface 136 to define a distance, Hb, between the bearer surface 130 and the base surface 136. In addition, the first and second sidewalls 132, 134 may be axially separated from each other such that the bearer surface 130 defines a width Wb extending axially along the second axis of rotation 114. It is to be appreciated that the width Wb may be constant or variable along the length of the bearer surface 130. In some embodiments, the bearer surface 130 may define a width Wb from about 2 mm to about 50 mm. It is also to be appreciated that in some embodiments, the first sidewall 132 and/or the second sidewall 134 may be perpendicular to base circumferential surface 136 or may be sloped or tapered with respect to the base circumferential surface 136.

As shown in FIG. 2, the pattern roll 108 is adjacent the anvil roll 106 so as to define a nip 126 between the pattern roll 108 and the anvil roll 106, and more particularly, to define a nip 126 between the bearer surface 130 and outer circumferential surface 116 of the anvil roll 106.

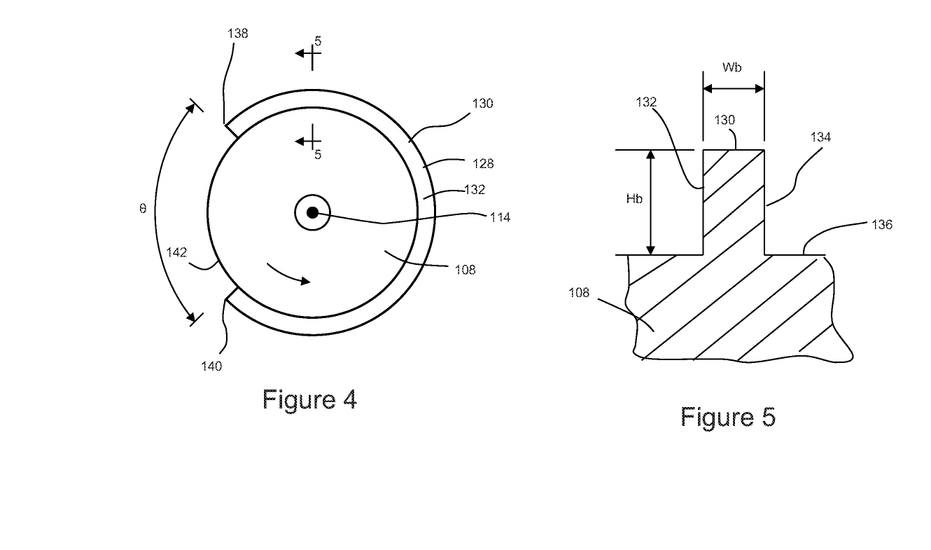

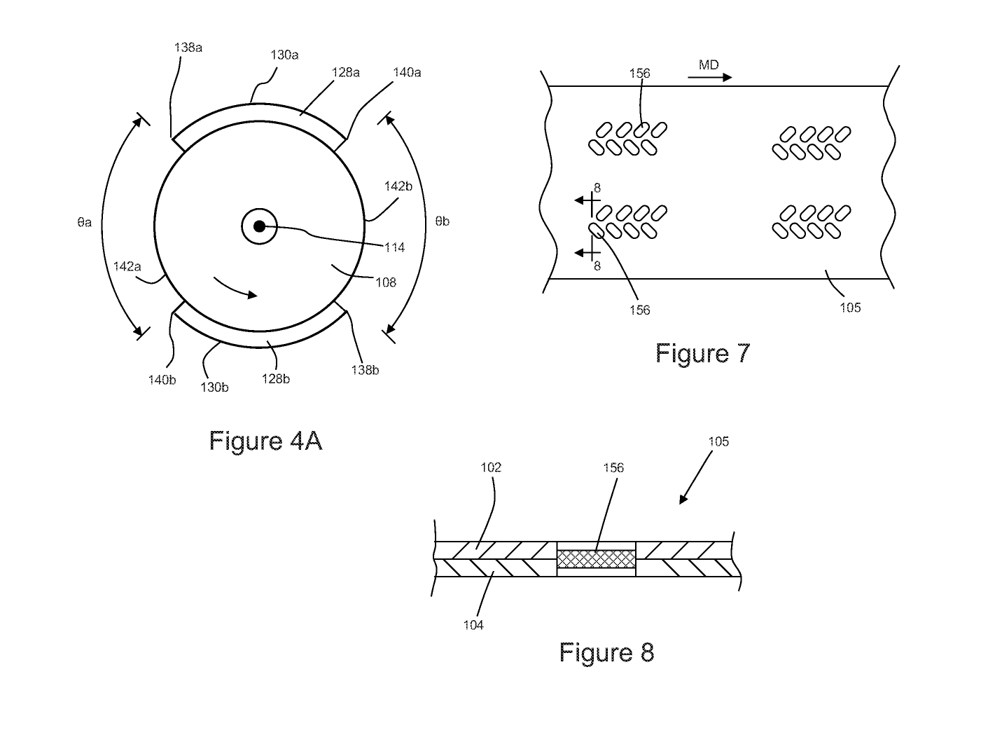

As shown in FIGS. 3 and 4, the bearer surface 130 extends from a first end 138 to a second end 140 to define a circumferential length extending around the second axis of rotation 114. More particularly, the bearer ring 128 extends for less than 360 degrees around the second axis of rotation 114 to define a circumferential gap region 142. As such, the circumferential gap region 142 extends θ degrees around the second axis of rotation between the first end 138 and the second end 140 of the bearer surface 130 and also extends, wherein 0 is less than 360 degrees. The circumferential gap region 142 extends axially along the length of the pattern roll 108. In turn, one or more of the pattern elements 118 and/or pattern surfaces 120 may be circumferentially offset from the bearer ring 132 and positioned in the circumferential gap region 142 represented in FIG. 3 by a cross-hatched, shaded area enclosed by a dashed lines.

As previously mentioned, the anvil roll 106 and the pattern roll 108 may be biased toward each other with a force F. Thus, as the anvil roll 106 and bonding roll 108 rotate to a position where a pattern element 118 contacts the anvil roll 106, a pattern pressure Pp is created between the pattern surface 120 and the outer circumferential surface 116 of the anvil roll 108. In addition, as the anvil roll 106 and bonding roll 108 rotate to a position where the bearer ring 128 contacts the anvil roll 106, a bearer pressure Pb is created between the bearer surface 130 and the outer circumferential surface 116 of the anvil roll 108. It is to be appreciated that the anvil roll 106 and the pattern roll 108 may be biased toward each other by applying force to either or both the anvil roll 106 and the pattern roll 108 in various ways, such as disclosed in U.S. Pat. No. 4,854,984. For example, as shown in FIG. 1, a supply source of pressurized air Pa may be connected with a regulator 144, which in turn, is connected with a pneumatic actuator 146. As such, the regulator 144 may be operated to adjust and control the pneumatic actuator 146, which in turn, operates to apply a force F on the pattern roll 108 toward the anvil roll 106. It is also to be appreciated that the pattern roll 108 and the anvil roll 106 may be configured to rotate such that the pattern surfaces 120 and the bearer surfaces 130 on the pattern roll 108 and the outer circumferential surface 116 of the anvil roll 106 move at the same speeds or different speeds.

As previously mentioned, the biasing of the pattern roll 108 and anvil roll 106 may be accomplished in various ways. In some embodiments, the bonding apparatus 100 is configured to define pattern pressures Pp and/or bearer pressures Pb above 60,000 PSI. In some embodiments, the bonding apparatus 100 is configured to define pattern pressures Pp and/or bearer pressures Pb from about 40,000 PSI to about 60,000 PSI. In some embodiments, the bonding apparatus 100 is configured to define pattern pressures Pp and/or bearer pressures Pb of about 40,000 PSI. In some embodiments, the bonding apparatus 100 is configured to define pattern pressures Pp and/or bearer pressures Pb of about 50,000 PSI. In some embodiments, the bonding apparatus 100 is configured to define pattern pressures Pp and/or bearer pressures Pb of about 60,000 PSI. It is also to be appreciated that the pattern roll 108 and/or the anvil roll 106 may be heated.

As shown in FIGS. 1, 7, and 8, during the bonding operation, the anvil roll 106 rotates in a direction A around the axis of rotation 112, and the pattern roll 108 rotates in direction B around the axis of rotation 114. The first substrate 102 and second substrate 104 advance in the machine direction MD between the pattern roll 108 and the anvil roll 106. More particularly, the first substrate 102 includes a first surface 148 and a second surface 150 opposite the first surface 148, and the second substrate 104 includes a first surface 152 and a second surface 154 opposite the first surface 152. As such, the first surface 148 of the first substrate 102 is contacted by the pattern roll 108, and the second surface 154 of the second substrate 104 is contacted by the anvil roll 106. And the second surface 150 of the first substrate 102 and the first surface 152 of the second substrate 104 contact each other. As first substrate 102 and second substrate 104 advance through the nip 126 between the pattern surface 120 of a pattern element 118 and the anvil roll 106, the pattern element 118 contacts the first substrate 102 and compresses the first substrate 102 and second substrate 104 between the pattern surface 120 of the pattern element 118 and the anvil roll 106. In turn, the nip pressure causes the first and second substrate material to yield. And the pattern surface 120 presses yielded material of the first and second substrates 102, 104 together to form a discrete bond region 156 between the first and second substrates 102, 104.

Thus, the apparatus 100 may form a laminate 105 including first and second substrates 102, 104 bonded together by discrete bond regions 156, without the use of adhesives. It is to be appreciated, however, that the bonding apparatus 100 may also be used in combination with adhesives. Although FIG. 1 shows the apparatus 100 bonding two substrates together, it is to be appreciated that the apparatus may bond more than two substrates together. In addition, it is to be appreciated that the apparatus may also be used to bond fibers of nonwoven together on a single substrate and/or emboss a pattern on a single substrate.

It is to be appreciated that the apparatuses and methods herein can be used to bond various types of substrates together. For example, in some embodiments the apparatus 100 may used to bond nonwoven substrates, such as for example, polypropylene nonwoven, polyethylene film, bi-component nonwoven or film, polyethylene terephthalate nonwoven or film. In some embodiments, the apparatuses and methods herein may be used to bond a substrate which includes a mixture of cellulosic fibers and polyethylene or polyethylene-polypropylene bicomponent fibers or particulate. In some embodiments, the substrates may have a basis weight of about 6 gsm to about 100 gsm. Other types of substrates can be sandwiched in between two layers of nonwovens or films.

It is to be appreciated that the apparatus 100 may be configured with various different configurations of pattern elements 118 and bearer rings 128. For example, as shown in FIGS. 2 and 3, the pattern roll 108 may be configured with a first bearer ring 128a and a second bearer ring 128b axially separated from each other along the axis of rotation 114. The pattern roll 108 may also include one or more groupings 119 of pattern elements 118. For example, as shown in FIGS. 2 and 3, the pattern roll 108 may be configured with a first grouping 119a and a second grouping 119b of pattern elements 118 separated from each other along the axis of rotation 114. It is also to be appreciated that pattern elements 118 may be positioned to be axially offset from the bearer rings 128. In some embodiments, the pattern elements 118 and/or pattern surfaces 120 may be positioned to be axially between (or inside) the bearer rings 128 and/or bearer surfaces 130, and in some embodiments, the bearer rings 128 and/or bearer surfaces 130 may be positioned to be axially between (or inside) the pattern elements 118 and/or pattern surfaces 120. In some embodiments, the pattern elements 118 and/or pattern surfaces 120 may axially aligned with the bearer rings 128 and/or bearer surfaces 130. It is also to be appreciated that in some embodiments, the anvil roll 106 may include one or more bearer rings 128.

It is also to be appreciated the pattern roll may include more than one bearer ring 128 defining more than one circumferential gap region 142 extending around the second axis of rotation 114. For example, FIG. 4A shows a side view of a pattern roll 108 including a first bearer ring 128a and a second bearer ring 128b, each extending for less than 360 degrees around the second axis of rotation 114. The first bearer ring 128a includes a first bearer surface 130a that extends from a first end 138a to a second end 140a, and the second bearer ring 128b includes a second bearer surface 130b that extends from a first end 138b to a second end 140b. The first and second bearer surfaces 130a, 130b are circumferentially offset from each other around the second axis of rotation 114 to define a first circumferential gap region 142a and a second circumferential gap region 142b. As such, the first circumferential gap region 142a extends θa degrees around the second axis of rotation 114 between the first end 138a of the first bearer surface 130a and the second end 140b of the second bearer surface 130b. In addition, the second circumferential gap region 142b extends θb degrees around the second axis of rotation 114 between the first end 138b of the second bearer surface 130b and the second end 140a of the first bearer surface 130a. It is to be appreciated that θa and θb may be equal or different and may be less than 360 degrees. In turn, one or more of the pattern elements 118 and/or pattern surfaces 120 may be circumferentially offset from the first and second bearer rings 128a, 128b and positioned in the first and second circumferential gap regions 142a, 142b.

It is also to be appreciated that the pattern roll 108 may be configured with pattern elements 118 and/or pattern surfaces 120 having different sizes and shapes. For example, in some embodiments, the pattern elements 118 and/or pattern surfaces 120 may have a perimeter that defines circular, square, rectangular, and various types of other shapes. For example, the pattern elements may have a perimeter that defines an elliptical shape. As such, in some embodiments, an elliptical shape pattern element may have a major axis of about 0.2 mm to about 20 mm. In some embodiments, the pattern elements may have pattern surfaces that define areas of about 0.04 mm² to about 400 mm². The pattern elements 118 and/or pattern surfaces 120 may also be arranged with various sized gaps between other, such as disclosed in U.S. Patent Publication No. 2014/0377513A1 and/or may also include channels of various sizes and shapes such a disclosed in U.S. Patent Publication No. 2014/0377506A1.

As previously mentioned, the pattern elements 118 and bearer rings 128 herein are configured to help reduce vibrations in the anvil roll 106 and/or pattern roll 108 caused by the alternating engagements of the pattern surfaces 120 and bearer surfaces 130 with the anvil roll 106 as the anvil roll 106 and pattern roll 108 rotate. As previously mentioned, the pattern roll 108 and the anvil roll 106 are biased toward each other with a force F. Thus, as the anvil roll 106 and bonding roll 108 rotate, pattern pressure Pp is created between the pattern surface 120 and the outer circumferential surface 116 of the anvil roll 106, and bearer pressure Pb is created between the bearer surface 130 and the outer circumferential surface 116 of the anvil roll 106. In some embodiments, the bearer pressure Pb may be about equal the pattern pressure Pp. In some embodiments, the bearer pressure Pb may be less than the pattern pressure Pp. And in some embodiments, the bearer pressure Pb may be greater than the pattern pressure Pp.

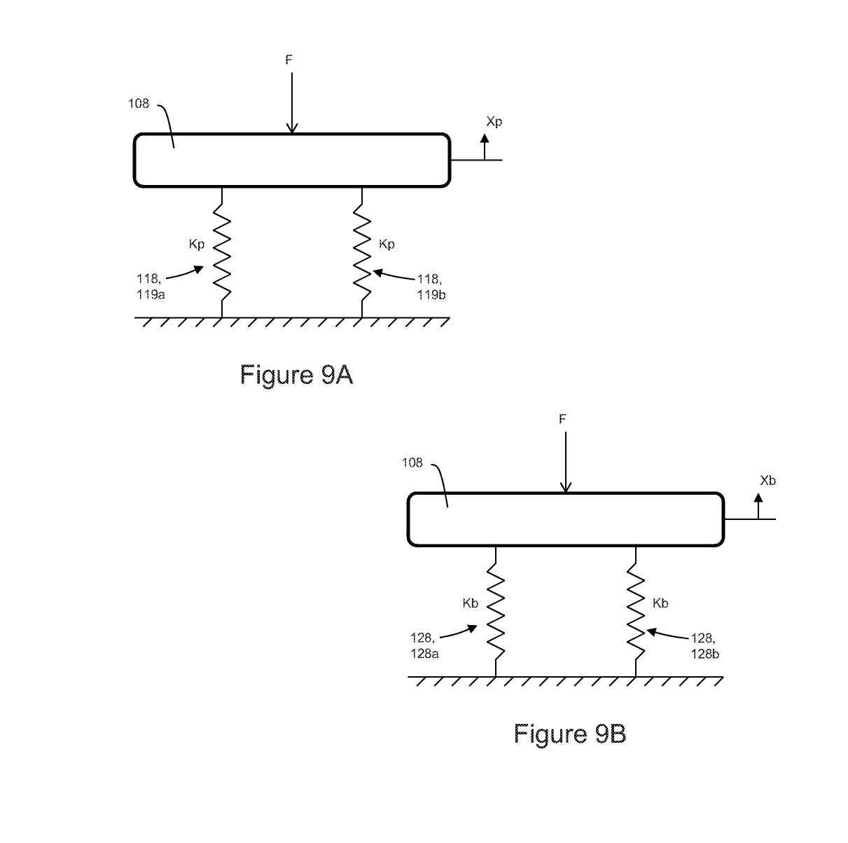

To help provide a visual context for the present discussion, FIGS. 9A and 9B are schematic representations of the pattern roll 108 from FIGS. 1 and 2 in terms of force F, displacements, and contact stiffness. During rotation of the pattern roll 108 and anvil roll 106, the force F displaces the pattern surface by a distance Xp when pattern elements 118 engage the anvil roll 106 such as shown in FIG. 9A. And the force F displaces the bearer surface 130 by a distance Xb when the bearer rings 128 engage the anvil roll 106 such as shown in FIG. 9B. The pattern elements 118 and bearer rings 128 are each configured to have a contact stiffness, Kp and Kb, respectively. As such, the distance Xp is defined by F/Kp, and the distance Xb is defined by F/Kb. It is to be appreciated that the contact stiffness Kb and/or Kp discussed herein may be determined in various ways. For example, one may calculate the contact stiffness Kb and/or Kp using Hertzian contact equations and/or finite element methodology.

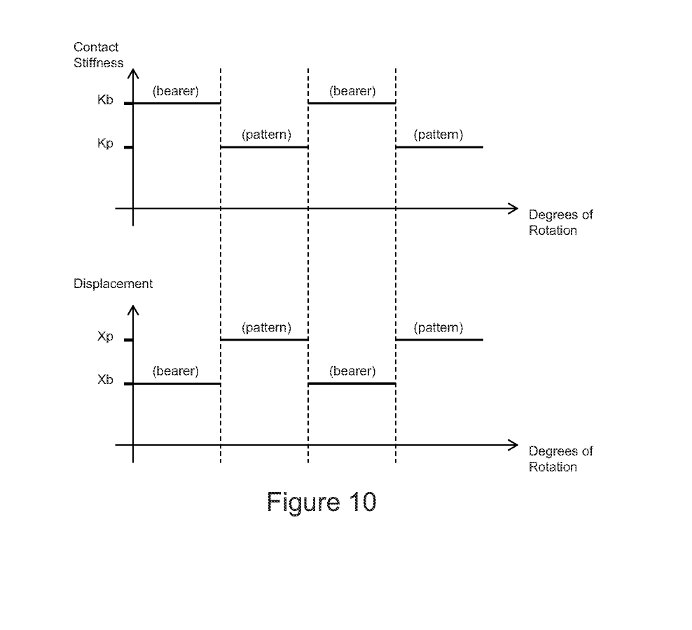

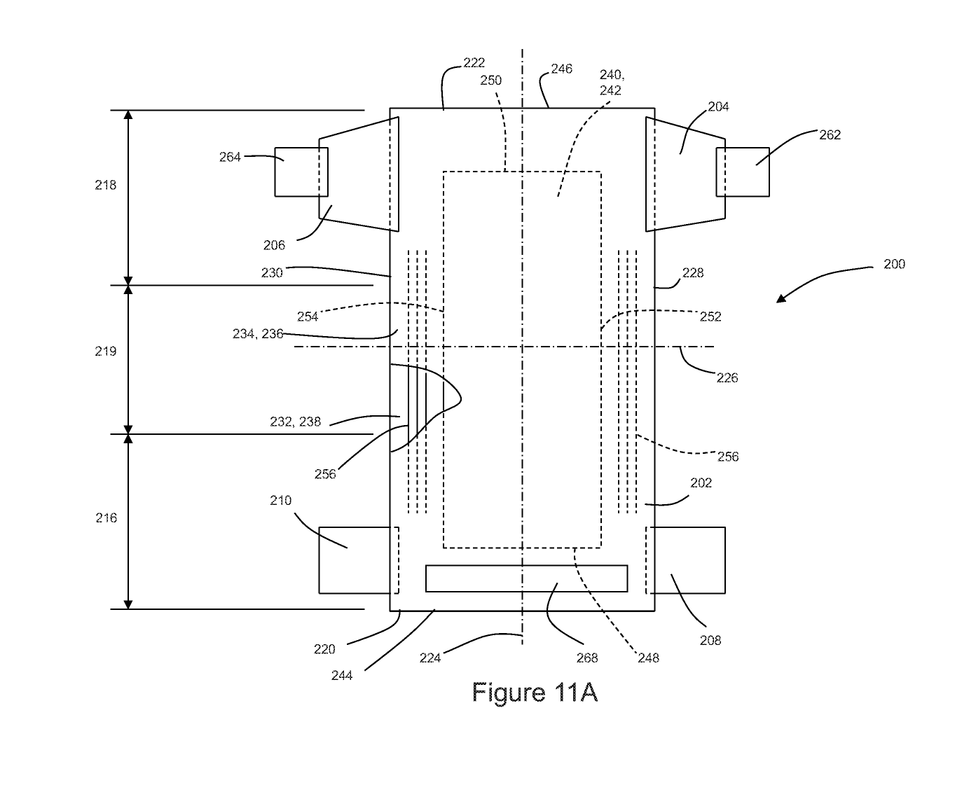

As graphically demonstrated in FIG. 10, the amplitude of the vibrations displacements Xb and Xp change as a function of the different contact stiffness Kb and Kp of the bearer rings 128 and the pattern elements 118 as the pattern roll 108 rotates. In the context of the above discussion, configuring the pattern elements 118 and bearer rings 128 to reduce the difference between the displacement distances Xb and Xp helps to reduce vibrations. Thus, pattern elements 118 and the bearer rings 128 may be configured such that the contact stiffness Kp of the pattern elements 118 is equal to or about equal to the contact stiffness Kb of the bearer rings 128. For example, in some embodiments, the bearer ring 128 and the pattern elements 118 may be constructed from the same material, such as for example, powder metal tool steels, such as CPM 10 V, tungsten carbides, and standard tool steels, such as a D2 or A2 steel. In turn, the pattern surface displacement distance Xp may be equal to or about equal to the bearer surface displacement distance Xb. And configuring the contact stiffness of the pattern elements 118 and bearer rings 128 to reduce the difference between the magnitude of the displacement distances Xp and Xb helps to reduce vibrations. In turn, relatively low vibrations may help reduce the frequency of pattern element deformations, including buckling, and/or failures and may help provide for more relatively more consistent and stronger bonds.

Although the tooling apparatus 100 discussed above is presented in the context of a bonding apparatus, it is to be appreciated that the tool rolls herein including tooling members and bearer rings with contact stiffness that are equal or about equal so as to reduce vibrations can be configured in various ways to perform various different operations, such as bonding, cutting, embossing, and/or activation of advancing substrates. For example, the tool roll may be configured as a knife roll with a tooling member in the form of a cutter member or blade, such a disclosed for example in U.S. Pat. Nos. 6,244,148; 7,861,756; and 7,777,094. In another example, the tool roll may be configured as an embossing roll or point bonding roll with a tooling member in the form of an embossing member or bonding member, such as disclosed in for example in U.S. Pat. Nos. 4,493,868; 5,620,779; and 5,798,167. In yet another example, the tool roll may be configured as an activation roll and/or bonding roll with a tooling member in the form of an activation member and/or bonding member, such as disclosed for example in European Patent Publication No. EP1635750B1.

As previously mentioned, the tooling apparatuses and methods herein may used to machine and/or modify various types of substrates and/or components used in the manufacture of different types of absorbent articles. To help provide additional context to the previous discussion of the process and apparatus embodiments, the following provides a general description of absorbent articles in the form of diapers that include substrates and/or components that may be bonded, embossed, cut, and/or activated with the methods and apparatuses disclosed herein.

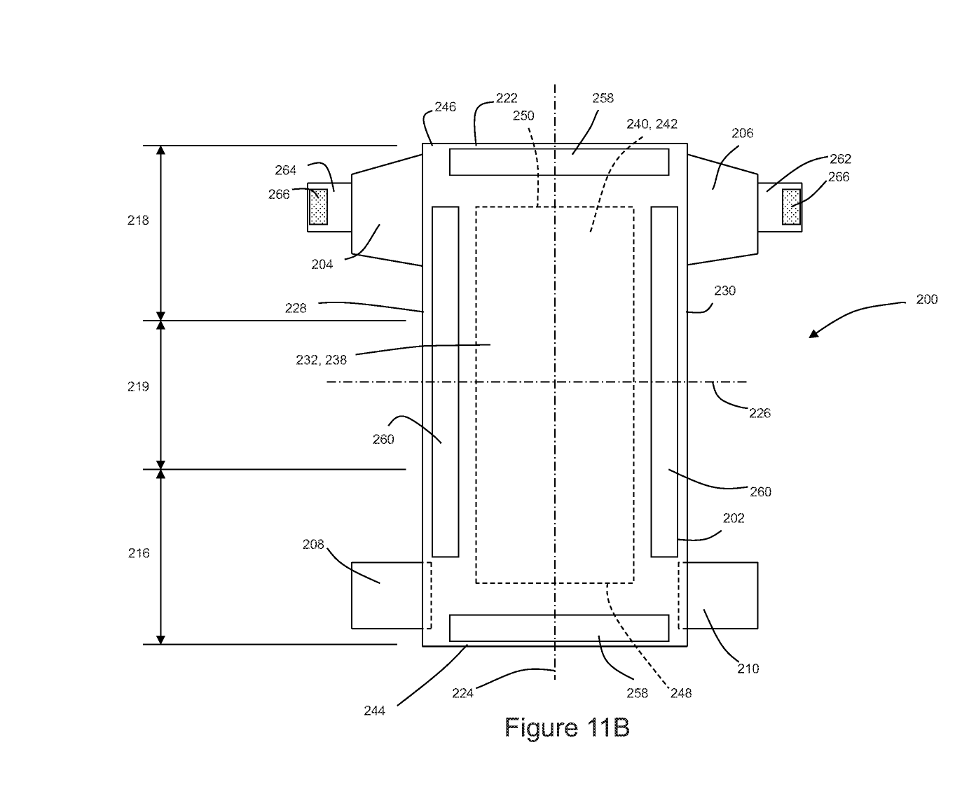

FIGS. 11A and 11B show an example of an absorbent article 100 that may be assembled in accordance with the methods and apparatuses disclosed herein. In particular, FIG. 11A shows one example of a plan view of an absorbent article configured as a taped diaper 200, with the portion of the diaper that faces away from a wearer oriented towards the viewer. And FIG. 11B shows a plan view of the diaper 200 with the portion of the diaper that faces toward a wearer oriented towards the viewer. The taped diaper 200 shown in FIGS. 11A and 11B includes a chassis 202, first and second rear side panels 204 and 206; and first and second front side panels 208 and 210.

As shown in FIGS. 11A and 11B, the diaper 200 and the chassis 202 each include a first waist region 216, a second waist region 218, and a crotch region 219 disposed intermediate the first and second waist regions. The first waist region 216 may be configured as a front waist region, and the second waist region 218 may be configured as back waist region. In some embodiments, the length of each of the front waist region, back waist region, and crotch region may be ⅓ of the length of the absorbent article 200. The absorbent article may also include a laterally extending front waist edge 220 in the front waist region 216 and a longitudinally opposing and laterally extending back waist edge 222 in the back waist region 218. To provide a frame of reference for the present discussion, the diaper 200 in FIGS. 11A and 11B are shown with a longitudinal axis 224 and a lateral axis 226. The longitudinal axis 224 may extend through a midpoint of the front waist edge 220 and through a midpoint of the back waist edge 222. And the lateral axis 226 may extend through a midpoint of a first longitudinal or right side edge 228 and through a midpoint of a second longitudinal or left side edge 230.

As shown in FIGS. 11A and 11B, the diaper 200 includes an inner, body facing surface 232, and an outer, garment facing surface 234. And the chassis 202 may include a backsheet 236 and a topsheet 238. The chassis 202 may also include an absorbent assembly 240, including an absorbent core 242, disposed between a portion of the topsheet 238 and the backsheet 236. As discussed in more detail below, the diaper 200 may also include other features, such as leg elastics and/or leg cuffs, an elastic waist region, and/or flaps, e.g., side panels and/or ears, to enhance the fits around the legs and waist of the wearer, to enhance the fit around the legs of the wearer.

As shown in FIGS. 11A and 11B, the periphery of the chassis 202 may be defined by the first longitudinal side edge 228, a second longitudinal side edge 230, a first laterally extending end edge 244 disposed in the first waist region 216, and a second laterally extending end edge 246 disposed in the second waist region 218. Both side edges 228 and 230 extend longitudinally between the first end edge 244 and the second end edge 246. As shown in FIG. 11A, the laterally extending end edges 244 and 246 may form a portion of the laterally extending front waist edge 220 in the front waist region 216 and a portion of the longitudinally opposing and laterally extending back waist edge 222 in the back waist region 218. When the diaper 200 is worn on the lower torso of a wearer, the front waist edge 220 and the back waist edge 222 may encircle a portion of the waist of the wearer. At the same time, the side edges 228 and 230 may encircle at least a portion of the legs of the wearer. And the crotch region 219 may be generally positioned between the legs of the wearer with the absorbent core 242 extending from the front waist region 216 through the crotch region 219 to the back waist region 218.

It is to also be appreciated that a portion or the whole of the diaper 200 may also be made laterally extensible. The additional extensibility may help allow the diaper 200 to conform to the body of a wearer during movement by the wearer. The additional extensibility may also help, for example, the user of the diaper 200, including a chassis 202 having a particular size before extension, to extend the front waist region 216, the back waist region 218, or both waist regions of the diaper 200 and/or chassis 202 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the diaper 200 may include a backsheet 236. The backsheet 236 may also define the outer surface 234 of the chassis 202. The backsheet 236 may be impervious to fluids (e.g., menses, urine, and/or runny feces) and may be manufactured in part from a thin plastic film, although other flexible liquid impervious materials may also be used. The backsheet 236 may prevent the exudates absorbed and contained in the absorbent core from wetting articles which contact the diaper 200, such as bedsheets, pajamas and undergarments. The backsheet 236 may also comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet may also comprise an elastomeric film. An example backsheet 236 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 236 may permit vapors to escape from the absorbent core (i.e., the backsheet is breathable) while still preventing exudates from passing through the backsheet 236. The size of the backsheet 236 may be dictated by the size of the absorbent core 242 and/or particular configuration or size of the diaper 200.

Also described above, the diaper 200 may include a topsheet 238. The topsheet 238 may also define all or part of the inner surface 232 of the chassis 202. The topsheet 238 may be compliant, soft feeling, and non-irritating to the wearer's skin. It may be elastically stretchable in one or two directions. Further, the topsheet 238 may be liquid pervious, permitting liquids (e.g., menses, urine, and/or runny feces) to penetrate through its thickness. A topsheet 238 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 238 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539.

As mentioned above, the diaper 200 may also include an absorbent assembly 240 that is joined to the chassis 202. As shown in FIGS. 11A and 11B, the absorbent assembly 240 may have a laterally extending front edge 248 in the front waist region 216 and may have a longitudinally opposing and laterally extending back edge 250 in the back waist region 218. The absorbent assembly may have a longitudinally extending right side edge 252 and may have a laterally opposing and longitudinally extending left side edge 254, both absorbent assembly side edges 252 and 254 may extend longitudinally between the front edge 248 and the back edge 250. The absorbent assembly 240 may additionally include one or more absorbent cores 242 or absorbent core layers. The absorbent core 242 may be at least partially disposed between the topsheet 238 and the backsheet 236 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprises primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 and 2004/0097895.

As previously mentioned, the diaper 200 may also include elasticized leg cuffs 256 and an elasticized waistband 258. It is to be appreciated that the leg cuffs 256 can be and are sometimes also referred to as leg bands, side flaps, barrier cuffs, elastic cuffs or gasketing cuffs. The elasticized leg cuffs 256 may be configured in various ways to help reduce the leakage of body exudates in the leg regions. Example leg cuffs 256 may include those described in U.S. Pat. Nos. 3,860,003; 4,909,803; 4,695,278; 4,795,454; 4,704,115; and U.S. Patent Publication No. 2009/0312730 A1.

The elasticized waistband 258 may provide improved fit and containment and may be a portion or zone of the diaper 200 that may elastically expand and contract to dynamically fit a wearer's waist. The elasticized waistband 258 may extend longitudinally inwardly from the waist edges 220, 222 of the diaper toward the lateral edges 248, 250 of the absorbent core 242. The diaper 200 may also include more than one elasticized waistband 258, for example, having one waistband 258 positioned in the back waist region 218 and one waistband 258 positioned in the front wait region 216, although other embodiments may be constructed with a single elasticized waistband 258. The elasticized waistband 258 may be constructed in a number of different configurations including those described in U.S. Pat. Nos. 4,515,595 and 5,151,092. In some embodiments, the elasticized waistbands 258 may include materials that have been “prestrained” or “mechanically prestrained” (subjected to some degree of localized pattern mechanical stretching to permanently elongate the material). The materials may be prestrained using deep embossing techniques as are known in the art. In some embodiments, the materials may be prestrained by directing the material through an incremental mechanical stretching system as described in U.S. Pat. No. 5,330,458. The materials are then allowed to return to their substantially untensioned condition, thus forming a zero strain stretch material that is extensible, at least up to the point of initial stretching. Examples of zero strain materials are disclosed in U.S. Pat. Nos. 2,075,189; 3,025,199; 4,107,364; 4,209,563; 4,834,741; and 5,151,092.

As shown in FIG. 11B, the chassis 202 may include longitudinally extending and laterally opposing side flaps 260 that are disposed on the interior surface 232 of the chassis 202 that faces inwardly toward the wearer and contacts the wearer. Each side flap may have a proximal edge. The side flaps may also overlap the absorbent assembly 240, wherein the proximal edges extend laterally inward of the respective side edges of the absorbent assembly 252 and 254. In some configurations, the side flaps may not overlap the absorbent assembly. It is to be appreciated that the side flaps may be formed in various ways, such as for example, by folding portions of the chassis 202 laterally inward, i.e., toward the longitudinal axis 224, to form both the respective side flaps and the side edges 228 and 230 of the chassis 202. In another example, the side flaps may be formed by attaching an additional layer or layers to the chassis at or adjacent to each of the respective side edges and of the chassis. Each of the side flaps may be joined to the interior surface 232 of the chassis and/or the absorbent assembly in side flap attachment zones in the front waist region 216 and in side flap attachment zones in the back waist region 218. The side flaps may extend to the same longitudinal extent as the absorbent article or alternatively the side flaps may have a longitudinal extent that is less than the absorbent article.

Taped diapers may be manufactured and provided to consumers in a configuration wherein the front waist region and the back waist region are not fastened, pre-fastened, or connected to each other as packaged, prior to being applied to the wearer. For example, the taped diaper 200 may be folded about a lateral centerline with the interior surface 232 of the first waist region 216 in surface to surface contact with the interior surface 232 of the second waist region 218 without fastening or joining the waist regions together. The rear side panels 204 and 206 and/or the front side panels 208 and 210 may also be folded laterally inward toward the inner surfaces 232 of the waist regions 216 and 218.

The diaper 200 may also include various configurations of fastening elements to enable fastening of the front waist region 216 and the back waist region 218 together to form a closed waist circumference and leg openings once the diaper is positioned on a wearer. For example, as shown in FIGS. 11A and 11B, the diaper 200 may include first and second fastening members 262, 264, also referred to as tabs, connected with the first and second rear side panels 204, 206, respectively. The diaper may also include first and second front side panels 208, 210, that may or may not include fastening members.

With continued reference to FIGS. 11A and 11B, each side panel 204, 206 and/or fastening member 262 and 264 may form a portion of or may be permanently bonded, adhered or otherwise joined directly or indirectly to the chassis 202 laterally inward from the side edge 228 and 230, in one of the front waist region 216 or the back waist region 218. Alternatively, the fastening members 262, 264 may form a portion of or may be permanently bonded, adhered or otherwise joined directly or indirectly to the first and second rear panels 204, 206 at or adjacent the distal edge of the panel and/or the first and second front side panels 208 and 210 at or adjacent the distal edge of the side panel. It is to be appreciated that the fastening members and/or side panels may be assembled in various ways, such as disclosed for example, in U.S. Pat. No. 7,371,302. The fastening members 262, 264 and/or side panels 204, 206, 208, 210 may also be permanently bonded or joined at or adjacent the side edges 228 and 230 of the chassis 202 in various ways, such as for example, by adhesive bonds, sonic bonds, pressure bonds, thermal bonds or combinations thereof, such as disclosed for example, U.S. Pat. No. 5,702,551.

Referring now to FIG. 11B, the first fastening member 262 and/or the second fastening member 264 may include various types of releasably engageable fasteners. The first and second fastening members 262 and/or 264 may also include various types of refastenable fastening structures. For example, the first and second fastening members 262 and 264 may include mechanical fasteners, 166, in the form of hook and loop fasteners, hook and hook fasteners, macrofasteners, buttons, snaps, tab and slot fasteners, tape fasteners, adhesive fasteners, cohesive fasteners, magnetic fasteners, hermaphrodidic fasteners, and the like. Some examples of fastening systems and/or fastening members 162, 164 are discussed in U.S. Pat. Nos. 3,848,594; 4,662,875; 4,846,815; 4,894,060; 4,946,527; 5,151,092; 5,221,274; 6,251,097; 6,669,618; 6,432,098; and U.S. Patent Publication Nos. 2007/0078427 and 2007/0093769.

As previously mentioned, the fastening members 262 and 264 may be constructed from various materials and may be constructed as a laminate structure. The fastening members 262 and 264 may also be adapted to releasably and/or refastenably engage or connect with another portion of the diaper 200. For example, as shown in FIG. 11A, the diaper 200 may include a connection zone 268, sometimes referred to as a landing zone, in the first waist region 216. As such, when the taped diaper 200 is placed on a wearer, the fastening members 262 and 264 may be pulled around the waist of the wearer and connected with the connection zone 268 in the first waist region 216 to form a closed waist circumference and a pair of laterally opposing leg openings. It is to be appreciated that the connection zone may be constructed from a separate substrate that is connected with the chassis 202 of the taped diaper. In some embodiments, the connection zone may be integrally formed as part of the backsheet 236 of the diaper 200 or may be formed as part of the first and second front panels 208, 210, such as described in U.S. Pat. Nos. 5,735,840 and 5,928,212.

In the context of the previous discussion, the apparatuses 100 and methods herein may be used to provide for the application of bonds 142 in patterns to substrates and components during the manufacture of an absorbent article 200. For example, bonds 142 may be applied in various patterns to portions of any of the topsheet 238, backsheet 236, absorbent core 140, leg cuffs 256, waist feature 258, side panels 204, 206, 208, 210, and fastening elements 262, 266 during the manufacture of an absorbent article 100. It is also to be appreciated that the apparatuses and methods herein may also be used to cut, emboss, and/or activate substrates and/or components such as topsheet 238, backsheet 236, absorbent core 140, leg cuffs 256, waist feature 258, side panels 204, 206, 208, 210, and fastening elements 262, 266.

Although the apparatuses and methods have been described in the context of the diaper 200 shown in FIGS. 11A, and 11B, it is to be appreciated that the methods and apparatuses herein may be used to assemble and/or bond, cut, emboss, and/or activate various substrates and/or components that can be used with various process configurations and/or absorbent articles, such as for example, disclosed in U.S. Pat. No. 7,569,039; U.S. Patent Publication Nos. 2005/0107764A1; 20120061016A1; 20120061015A1; 2013/0255861A1; 2013/0255862A1; 2013/0255863A1; 2013/0255864A1; and 2013/0255865A1, all of which are incorporated by reference herein. For example, the bonding apparatuses and methods herein can be used to apply tack-down bonds on leg cuffs, such as described in U.S. Patent Publication No. 2013/0255865A1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An apparatus for bonding substrates, the apparatus comprising:
a frame;
an anvil roll rotatably connected with the frame and adapted to rotate about a first axis of rotation, the anvil roll comprising an outer circumferential surface;
a bonding roll rotatably connected with the frame and adapted to rotate about a second axis of rotation, wherein the first axis of rotation is parallel with the second axis of rotation, the bonding roll comprising:
a bearer ring comprising a bearer surface extending from a first end to a second end to define a circumferential length, the bearer ring extending for less than 360 degrees around the second axis of rotation to define a circumferential gap region between the first end and the second end, the bearer ring comprising a contact stiffness, Kb;
a pattern element comprising a pattern surface circumferentially offset from the bearer ring and positioned in the circumferential gap region, the pattern element comprising a contact stiffness Kp;
wherein the bonding roll and the anvil roll are biased toward each other with a force F, such that as the anvil roll and bonding roll rotate, a pattern pressure Pp is created between the pattern surface and the outer circumferential surface of the anvil roll and a bearer pressure Pb is created between the bearer surface and the outer circumferential surface of the anvil roll; and
wherein the force F displaces the pattern surface by a distance Xp, wherein Xp is defined by F/Kp and wherein the force F displaces the bearing surface by a distance Xb, wherein Xb is defined by F/Kb; and
wherein Kp is about equal to Kb.

2. The apparatus of claim 1, wherein the bearer surface defines a width extending axially along the second axis of rotation.

3. The apparatus of claim 2, wherein the width of the bearer surface varies along the circumferential length.

4. The apparatus of claim 1, wherein the pattern surface is axially displaced from the bearer surface.

5. The apparatus of claim 1, wherein the bearer pressure Pb is about equal the pattern pressure Pp.

6. The apparatus of claim 1, wherein the bearer ring comprises a first bearer ring and a second bearer ring, wherein the first bearer ring is axially displaced from the second bearer ring.

7. The apparatus of claim 6, wherein the pattern surface is positioned axially between the first bearer ring and the second bearer ring.

8. An apparatus for bonding substrates, the apparatus comprising:
a frame;
an anvil roll rotatably connected with the frame and adapted to rotate about a first axis of rotation, the anvil roll comprising an outer circumferential surface;
a bonding roll rotatably connected with the frame and adapted to rotate about a second axis of rotation, wherein the first axis of rotation is parallel with the second axis of rotation, the bonding roll comprising:
a first bearer ring comprising a first bearer surface extending from a first end to a second end to define a circumferential length, the first bearer ring extending for less than 360 degrees around the second axis of rotation;
a second bearer ring comprising a second bearer surface extending from a first end to a second end to define a circumferential length, the second bearer ring extending for less than 360 degrees around the second axis of rotation; the second end of the first bearer surface circumferentially offset from the first end of the second bearer surface to define a circumferential gap region between the first end and the second end;
a pattern element comprising a pattern surface circumferentially offset from the first and second bearer rings and positioned in the circumferential gap region;
wherein the bonding roll and the anvil roll are biased toward each other with a force F, such that as the anvil roll and bonding roll rotate, a pattern pressure Pp is created between the pattern surface and the outer circumferential surface of the anvil roll and bearer pressures Pb are created between the first and second bearer surfaces and the outer circumferential surface of the anvil roll; and
wherein the force F displaces the pattern surface by a distance Xp;
wherein the force F displaces the first bearer surface and the second bearer surface by a distance Xb; and
wherein Xb is about equal to Xp.

9. The apparatus of claim 8, wherein the first bearer ring comprises a contact stiffness, Kb, wherein Xb is defined by F/Kb.

10. The apparatus of claim 8, wherein the pattern element comprises a contact stiffness, Kp, wherein Xp is defined by F/Kp.

11. The apparatus of claim 8, wherein the first bearer ring is axially aligned with the second bearer ring.

12. The apparatus of claim 8, wherein the first bearer surface defines a width extending axially along the second axis of rotation.

13. The apparatus of claim 12, wherein the width of the first bearer surface varies along the circumferential length.

14. The apparatus of claim 8, wherein the pattern surface is axially displaced from the first bearer surface and the second bearer surface.

15. The apparatus of claim 14, wherein the pattern surface is positioned axially between the first bearer ring and the second bearer ring.

16. The apparatus of claim 8, wherein the bearer pressure Pb is about equal to the pattern pressure Pp.

17. A method of bonding substrates, the method comprising the steps of:
providing an anvil roll adapted to rotate about a first axis of rotation, the anvil roll comprising an outer circumferential surface;
providing a bonding roll adapted to rotate about a second axis of rotation adjacent the anvil roll, wherein the first axis of rotation is parallel with the second axis of rotation, the bonding roll comprising a bearer surface and a pattern surface;
rotating the anvil roll and the bonding roll in opposite directions such that the pattern surface and the bearer surface alternatingly contact the outer circumferential surface of the anvil roll;
biasing the anvil roll and the bonding roll toward each other with a force, F;
displacing the pattern surface by a distance Xp with the force F;
displacing the bearer surface by a distance Xb with the force F, wherein Xb is about equal to Xp; and
advancing a first substrate and a second substrate in a machine direction between the bonding roll and the anvil roll to form a discrete bond region between the first and second substrates.

18. The method of claim 17, wherein the bearer surface extends from a first end to a second end to define a circumferential length, the bearer surface extending for less than 360 degrees around the second axis of rotation to define a circumferential gap region between the first end and the second end.

19. The method of claim 18, wherein the pattern surface is circumferentially offset from the bearer surface and positioned in the circumferential gap region.

20. The method of claim 18, further comprising a pattern element comprising the pattern surface and a bearer ring further comprising the bearer surface, the pattern element comprising a contact stiffness Kp, the bearer ring comprising a contact stiffness, Kb, wherein Kp is about equal to Kb.
